(19) United States
(12) Patent Application Publication
(10) Pub. No.: US 2026/0294185 A1
(43) Pub. Date: Oct. 1, 2026

(54) SCENTED VACUUM FILTER PAD

(71) Applicant: Brittany Grillo, Rockford, MI (US)

(72) Inventor: Brittany Grillo, Rockford, MI (US)

(21) Appl. No.: 19/090,322

(22) Filed: Mar. 25, 2025

(51) Int. Cl. A47L 7/04; A47L 9/12; A61L 9/12

(52) U.S. Cl. CPC A47L 7/04; A47L 9/12; A61L 9/12

(57) ABSTRACT

A vacuum filter fragrance pad includes a circular pad made of semisynthetic breathable felt material with a thickness of approximately 2 mm. The pad is infused with a scented oil by soaking in fragrance oils for up to 48 hours. The pad is sized to fit within a vacuum cleaner's filter compartment and is designed to be placed directly on top of the vacuum's foam or HEPA filter. The scented pad releases fragrance during vacuum operation, acting as a moving air freshener without clogging filters or damaging vacuum components. The pad is reusable and provides fragrance for up to 30 days before needing replacement.

### BACKGROUND

### 1. Field of the Invention

The present disclosure relates to air freshening devices for vacuum cleaners, and more particularly to a scented filter pad designed to be placed directly onto a vacuum's filter to provide fragrance during vacuum operation.

### 2. Description of the Prior Art

Vacuum cleaners have long been an essential household appliance for maintaining clean and hygienic living spaces. These devices operate by creating suction to remove dust, debris, and other particulates from floors and surfaces. As vacuum technology has advanced, manufacturers have sought to improve not only the cleaning efficiency but also the overall user experience.

One aspect of vacuum cleaner operation that has received attention is the quality of air exhausted from the device during use. Traditional vacuum cleaners often emit a characteristic odor, which can be unpleasant for users and may even reintroduce unwanted particles into the air. This issue has led to various attempts to address the exhaust air quality, including the development of filtration systems and the incorporation of air freshening mechanisms.

However, existing solutions for improving vacuum cleaner exhaust air have faced several challenges. Many approaches require complex modifications to the vacuum cleaner design or the use of specialized components, which can increase manufacturing costs and complexity. Additionally, some methods for introducing fragrances or deodorizers into the exhaust air stream may interfere with the vacuum's primary cleaning function or reduce its overall efficiency.

There remains a need for a simple, effective, and user-friendly solution to enhance the quality of air exhausted from vacuum cleaners during operation. Ideally, such a solution would be compatible with existing vacuum cleaner designs, easy to implement, and capable of providing a pleasant scent without compromising the device's cleaning performance or filtration capabilities.

### SUMMARY

According to an aspect of the present disclosure, a vacuum filter fragrance pad is provided. The vacuum filter fragrance pad includes a semisynthetic breathable felt material which may optionally be shaped into a disc. The fragrance pad has a thickness of 2 millimeters. The semisynthetic breathable felt material is infused with a fragrance oil blend.

According to other aspects of the present disclosure, the vacuum filter fragrance pad may include one or more of the following features. The semisynthetic breathable felt material may comprise a blend of natural and synthetic fibers. The fragrance oil blend may be infused into the semisynthetic breathable felt material through a soaking process lasting up to 48 hours. The vacuum filter fragrance pad may be sized to fit within a vacuum cleaner filter compartment. The vacuum filter fragrance pad may be reusable. The vacuum filter fragrance pad may be designed to release fragrance for up to 30 days when used in a vacuum cleaner.

According to another aspect of the present disclosure, a method of manufacturing a vacuum filter fragrance pad is provided. The method includes selecting a semisynthetic breathable felt material, cutting the material into a disc shape with a thickness of 2 millimeters, soaking the disc in a fragrance oil blend for up to 48 hours, and compressing the soaked disc to remove excess liquid while retaining an optimal amount of fragrance oil.

According to other aspects of the present disclosure, the method may include one or more of the following features. The method may include compressing 50 or fewer discs at a time to ensure even oil distribution. The method may include compressing the discs to a specific pressure for about 3 seconds, then turning the discs over and repeating the compression. The method may include packaging the compressed discs in protective packaging to preserve freshness and fragrance.

For a more complete understanding, reference is made to the following detailed description and accompanying drawings. In the drawings, like reference characters refer to like parts throughout the views in which:

### BRIEF DESCRIPTION OF THE DRAWINGS

FIG. 1 illustrates a top orthogonal view of a vacuum filter fragrance pad in accordance with an embodiment of the disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to a vacuum filter fragrance pad that may be used to impart a pleasant scent while vacuuming. In some cases, the vacuum filter fragrance pad may be placed directly on a vacuum's existing filter to release fragrance during operation. The vacuum filter fragrance pad may provide an efficient and convenient way to freshen the air while cleaning. In some implementations, the vacuum filter fragrance pad may be reusable and long-lasting. The vacuum filter fragrance pad may allow a vacuum cleaner to function as a moving air freshener, distributing scent throughout a space as vacuuming occurs. In certain cases, the vacuum filter fragrance pad may be designed to avoid clogging or damaging vacuum components while still effectively releasing fragrance.

Based upon the foregoing disclosure, it is seen that the present disclosure provides a vacuum filter fragrance pad that may offer advantages over traditional vacuum freshening products. The vacuum filter fragrance pad may allow for air freshening without the need for separate products or additional steps during cleaning. The pad may also avoid issues associated with powder or bead-based fresheners, such as clogging filters or damaging internal vacuum components. Additionally, the reusable nature of the pad may provide a more sustainable option compared to single-use freshening products.

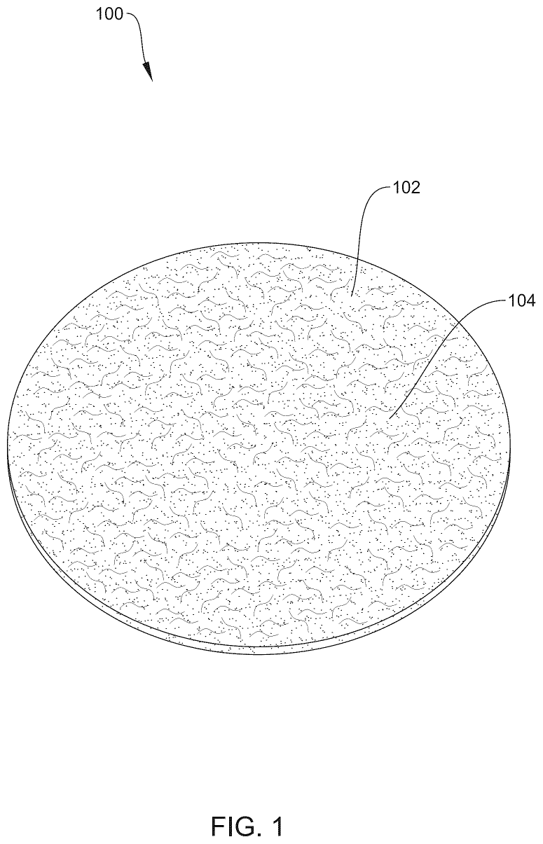

FIG. 1 illustrates a scented pad 100 comprising a pad 102 infused with a scented oil 104. The pad 102 may be composed of a semisynthetic breathable felt material. Semisynthetic breathable felt material refers to a non-woven textile material that combines both natural and man-made fibers to create a porous structure that allows air to pass through while maintaining structural integrity. This material is characterized by its ability to absorb and retain liquids such as fragrance oils while still permitting airflow, making it ideal for applications requiring both fragrance retention and air circulation. In some cases, the semisynthetic breathable felt material may include a blend of natural and synthetic fibers. The pad 102 may have a thickness of approximately 2 mm.

In some cases, the pad 102 may incorporate different ratios of natural and synthetic fibers. For example, the pad 102 may comprise 70% wool and 30% polyester, which may enhance fragrance retention. The natural wool fibers provide excellent absorption properties for the scented oils, while the synthetic polyester adds structural stability. Alternatively, the pad 102 may comprise 70% polyester and 30% rayon, which may improve durability and resistance to breakdown from fragrance oils. The semisynthetic nature of the material allows for customization of these ratios to optimize both performance and longevity of the fragrance pad.

The pad 102 may, in some cases, incorporate bamboo fibers. The inclusion of bamboo fibers may provide enhanced antimicrobial properties to the scented pad 100.

In some implementations, the pad 102 may include activated charcoal fibers. The addition of activated charcoal fibers to the felt blend may provide additional odor absorption capabilities.

The thickness of the pad 102 may vary in different implementations. The pad 102 is preferably 2 mm thick. For example, the pad 102 may have a thickness of 1.5 mm, which may be suitable for vacuums with limited filter space. Alternatively, the pad 102 may have a thickness of 3 mm, which may potentially provide longer-lasting fragrance release.

In some cases, the pad 102 may have a variable thickness across its surface. For example, the pad 102 may have a thickness of 2.5 mm in the center, tapering to 1.5 mm at the edges. This variable thickness may optimize airflow and fragrance distribution.

The pad 102 may, in some implementations, have a multi-layer design. Each layer of the multi-layer design may have different densities or may be composed of different materials.

In some implementations, the pad 102 may be formed in various shapes to accommodate different vacuum filter designs or user preferences. While a circular disc shape may be preferable for its versatility and ease of placement on most vacuum filters, other shapes may be utilized. For example, the pad 102 may be rectangular, square, oval, or even custom-shaped to fit specific vacuum models. In some cases, the pad 102 may be triangular or star-shaped to provide increased surface area for fragrance release. Alternatively, the pad 102 may be designed with a central cutout or in a ring shape to allow for better airflow in certain vacuum configurations. Some implementations may feature a pad 102 with rounded corners or a hexagonal shape for improved fit in particular filter housings. Despite these various possibilities, a circular disc shape may be preferred for its simplicity, uniform fragrance distribution, and compatibility with a wide range of vacuum filter designs.

In some cases, the pad 102 may incorporate a central hole or multiple smaller holes. These holes may accommodate different vacuum filter designs.

The pad 102 may, in some implementations, include perforated patterns. These perforated patterns may enhance airflow and fragrance distribution.

In some cases, the pad 102 may include tabs or extensions. These tabs or extensions may facilitate easier placement and removal of the scented pad 100.

Based upon the foregoing disclosure, it is seen that the present disclosure discloses a scented pad for vacuum filters that offers versatility in design and composition. The scented pad surpasses the prior art because of its ability to incorporate various materials for enhanced functionality, its adaptable thickness for different vacuum models, and its potential for optimized fragrance distribution and airflow. The scented pad also provides improved ease of use through design features such as tabs or extensions, making it a more user-friendly solution for vacuum fragrance applications.

FIG. 1 illustrates a scented pad 100 comprising a pad 102 infused with a scented oil 104. The scented oil 104 may be a custom blend of fragrance oils selected for their long-lasting and odor-masking properties. In some cases, the scented oil 104 may include a mixture of essential oils and fragrance oils to create unique aromatic profiles.

The infusion process for the scented oil 104 into the pad 102 may involve several techniques. In some cases, the pad 102 may be fully immersed in the scented oil 104 for a period ranging from 24 to 72 hours, preferably up to 48 hours. This extended soaking time may allow for thorough saturation of the pad 102 with the scented oil 104, ensuring a long-lasting fragrance release during vacuum operation.

In some implementations, a pressurized infusion system may be employed to introduce the scented oil 104 into the pad 102. This method may potentially reduce the soaking time while maintaining a high level of oil saturation within the pad 102. The pressurized system may allow for more precise control over the infusion process, potentially resulting in a more uniform distribution of the scented oil 104 throughout the pad 102.

A multi-stage infusion process may be utilized in some cases to create complex scent profiles. This approach may involve infusing the pad 102 with different layers of fragrances sequentially. For example, a base note fragrance may be infused first, followed by middle notes, and finally top notes. This layered approach may result in a more sophisticated and nuanced scent experience as the fragrance evolves during vacuum use.

In some implementations, ultrasonic technology may be employed to enhance the penetration of the scented oil 104 into the fibers of the pad 102. Ultrasonic waves may help break down the oil molecules into smaller particles, potentially allowing for deeper and more uniform penetration into the felt material. This technique may result in improved fragrance retention and a more consistent scent release over time.

The scented oil 104 may be formulated to provide various benefits beyond simply masking odors. In some cases, the scented oil 104 may include antimicrobial components, such as tea tree oil or eucalyptus oil, which may help reduce bacterial growth within the vacuum filter. Other formulations may incorporate calming scents like lavender or chamomile, potentially creating a more relaxing environment during cleaning activities.

Different fragrance blends may be created to suit various preferences and applications. For example, citrus-based scents may be formulated for their energizing and fresh qualities, while woody or musky scents may be designed for a more subtle and sophisticated aroma. Seasonal blends, such as pine or cinnamon for winter, or floral scents for spring, may also be developed to provide users with a variety of options throughout the year.

Based upon the foregoing disclosure, it is seen that the present disclosure provides a versatile and effective method for infusing vacuum filter pads with long-lasting fragrances. The scented pad 100 offers advantages over traditional vacuum fresheners by providing a consistent and controllable scent release without compromising vacuum performance. The various infusion techniques and fragrance blend options allow for customization and optimization of the scent experience, potentially enhancing the overall cleaning process for users.

The manufacturing process for the vacuum filter fragrance pad involves several key steps, including material selection, fragrance infusion, and compression techniques.

Material selection begins with choosing a semisynthetic breathable felt material. This material may be composed of a blend of natural and synthetic fibers, such as wool and polyester. The specific ratio of natural to synthetic fibers may be adjusted to optimize fragrance retention and durability. In some cases, the felt material may incorporate additional fibers such as bamboo or activated charcoal to enhance antimicrobial properties or odor absorption capabilities.

The fragrance infusion process involves soaking the felt pads in a custom blend of fragrance oils. The pads may be fully immersed in the fragrance oil mixture for a period ranging from 24 to 72 hours, depending on the desired fragrance intensity. In some cases, a pressurized infusion system may be used to potentially reduce soaking time while maintaining saturation. Alternatively, a multi-stage infusion process with different fragrance layers may be implemented to create a more complex scent profile. In some embodiments, ultrasonic technology may be utilized to enhance oil penetration into the felt fibers.

Following the infusion process, the pads undergo a compression step to remove excess liquid and optimize fragrance retention. This compression step may include compressing 50 or fewer discs at a time to ensure even oil distribution throughout each pad. The method may include applying a specific pressure to a first side of the discs for approximately 3 seconds, then turning the discs over and repeating the compression process on the second side for the same duration. This controlled compression technique helps achieve optimal fragrance retention while removing any excess oil that might potentially damage vacuum components.

The compression technique may involve several variations:
1. Vacuum-assisted compression: In some cases, a vacuum-assisted compression process may be employed to more precisely control oil retention within the felt pad. This technique may involve placing the infused pads in a sealed chamber and applying a controlled vacuum to extract excess oil while maintaining a specific level of saturation.
2. Heated compression: The compression step may incorporate heat in some embodiments. A heated compression process may potentially alter the felt structure for improved fragrance holding capacity. The temperature and duration of heating may be carefully controlled to achieve the desired effect without compromising the integrity of the felt material or the fragrance oils.
3. Variable pressure compression: In some cases, the compression step may apply variable pressure across the pad surface. This technique may create zones of different fragrance intensities within a single pad. For example, the center of the pad may receive higher pressure to create a more concentrated fragrance core, while the edges may receive less pressure for a more gradual release of scent.

The compression process may be performed in batches, with a specific number of pads compressed together to ensure consistent oil retention across all pads. The pressure applied during compression, as well as the duration of compression, may be carefully calibrated to achieve the optimal balance of fragrance retention and pad performance.

Based upon the foregoing disclosure, it is seen that the present disclosure provides a vacuum filter fragrance pad with several advantages over traditional air freshening products. The manufacturing process allows for precise control of fragrance infusion and retention, resulting in a product that releases a consistent and long-lasting scent without compromising vacuum performance. The use of semisynthetic breathable felt material and specialized compression techniques enables the pad to retain fragrance effectively while maintaining proper airflow through the vacuum's filtration system. Additionally, the flexibility in material composition and fragrance infusion methods allows for customization of scent profiles and pad properties to suit various applications and user preferences.

FIG. 1 illustrates a scented pad 100 comprising a pad 102 infused with a scented oil 104. The pad 102 may be made of a semisynthetic breathable felt material, which allows for optimal retention and release of the scented oil 104. In some cases, the pad 102 may have a thickness of approximately 2 mm, providing sufficient volume to hold the scented oil 104 while maintaining a low profile for placement within a vacuum cleaner.

The scented pad 100 may be designed to be placed directly on top of a vacuum's foam or HEPA filter. The circular shape of the pad 102 allows for easy fitting onto most standard vacuum filters. In some cases, the pad 102 may be cut in half to accommodate smaller filter sizes or unique vacuum designs.

When the vacuum is in operation, air flows through the scented pad 100, causing the gradual release of the scented oil 104. The semisynthetic breathable felt material of the pad 102 may allow for controlled diffusion of the scented oil 104, ensuring a consistent fragrance release over time. The airflow generated by the vacuum may help distribute the fragrance throughout the room being cleaned.

The interaction between the pad 102 and the scented oil 104 may be optimized to provide long-lasting fragrance without compromising the vacuum's performance. The breathable nature of the felt material may allow air to pass through while retaining the scented oil 104, preventing clogging of the vacuum's filtration system.

In some cases, the scented pad 100 may remain effective for up to 30 days, depending on the frequency of vacuum use and the specific fragrance formulation. The gradual release of the scented oil 104 may ensure that the fragrance remains noticeable throughout the pad's lifespan without overwhelming the user or diminishing too quickly.

Based upon the foregoing disclosure, it is seen that the present disclosure provides a vacuum filter fragrance pad that offers several advantages over traditional vacuum freshening methods. The scented pad 100 may provide a more consistent and long-lasting fragrance compared to powders or beads, while avoiding potential damage to vacuum components. The design may allow for easy installation and removal, enhancing user convenience. Additionally, the scented pad 100 may serve as a moving air freshener, distributing pleasant scents throughout the home during the vacuuming process without leaving residue on surfaces or potentially harmful particles in the air.

Based upon the foregoing disclosure, it is seen that the present disclosure provides several advantages and benefits for vacuum filter fragrance pads. The semisynthetic breathable felt material allows for optimal airflow through the vacuum's filtration system while effectively retaining fragrance oils. This design enables the pad to release pleasant scents during vacuum operation without compromising the vacuum's performance or suction power.

The fragrance infusion process, involving soaking the pads for up to 48 hours, ensures thorough saturation and long-lasting scent release. This method may result in fragrance that lasts up to 30 days, providing extended freshness compared to traditional air freshening products.

The compression technique used in manufacturing helps achieve the ideal balance of fragrance retention without over-saturating the pad. This process may prevent excess liquid from potentially damaging vacuum components or affecting filtration efficiency.

The vacuum filter fragrance pads may be compatible with various vacuum models, as they can be placed directly on top of foam or HEPA filters. In some cases, the pads may be cut to fit specific filter sizes, enhancing their versatility across different vacuum brands and models.

Unlike powder-based or bead air fresheners, these fragrance pads do not introduce particulate matter into the vacuum system. This design may help prevent clogging of filters or damage to internal vacuum components, potentially extending the lifespan of the vacuum cleaner.

The reusable nature of the fragrance pads offers an environmentally friendly alternative to disposable air freshening products. Users may simply replace the pad when the fragrance dissipates, reducing waste compared to single-use freshening solutions.

The vacuum filter fragrance pads may serve as moving air fresheners, distributing scent evenly throughout a space as the vacuum operates. This feature may enhance the overall cleaning experience by combining odor elimination with pleasant fragrance distribution.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The exemplary embodiment was chosen and described in order to best explain the principles of the present disclosure and its practical application, to thereby enable others skilled in the art to best utilize the present disclosure and various embodiments with various modifications as are suited to the particular use contemplated.

What is claimed is:

1. A vacuum filter fragrance pad, comprising:
a pad made of semisynthetic breathable felt material; and
a scented oil infused into the pad,
wherein the pad is configured to be placed directly onto a vacuum's filter to provide fragrance during vacuum operation.

2. The vacuum filter fragrance pad of claim 1, wherein the pad is circular.

3. The vacuum filter fragrance pad of claim 1, wherein the pad has a thickness of 2 mm.

4. The vacuum filter fragrance pad of claim 1, wherein the semisynthetic breathable felt material comprises a blend of natural and synthetic fibers.

5. The vacuum filter fragrance pad of claim 4, wherein the natural fibers comprise wool and the synthetic fibers comprise polyester.

6. The vacuum filter fragrance pad of claim 1, wherein the scented oil comprises a blend of essential oils and fragrance oils.

7. The vacuum filter fragrance pad of claim 1, wherein the pad is reusable and configured to be replaced when no longer fragrant.

8. A method of manufacturing a vacuum filter fragrance pad, comprising:
selecting a pad made of semisynthetic breathable felt material;
soaking the pad in a scented oil for a predetermined time period; and
compressing the soaked pad to release excess scented oil,
wherein the resulting vacuum filter fragrance pad is configured to be placed directly onto a vacuum's filter.

9. The method of claim 8, wherein the pad is circular.

10. The method of claim 8, wherein the predetermined time period for soaking the pad in the scented oil is between 24 to 48 hours.

11. The method of claim 8, wherein compressing the soaked pad comprises applying pressure to release excess scented oil while retaining an optimal amount of scented oil in the pad.

12. The method of claim 11, wherein compressing the soaked pad comprises applying pressure to groups of 50 or fewer pads at a time.

13. The method of claim 12, wherein compressing the soaked pad further comprises:
applying pressure to a first side of the pad for a predetermined duration;
turning the pad over; and
applying pressure to a second side of the pad for the predetermined duration.

14. The method of claim 8, wherein the scented oil comprises a blend of essential oils and fragrance oils.

15. A vacuum cleaning system, comprising:
a vacuum cleaner having a filter; and
a fragrance pad positioned on the filter, the fragrance pad comprising a pad made of semisynthetic breathable felt material infused with a scented oil,
wherein the fragrance pad is configured to release fragrance during operation of the vacuum cleaner without compromising the vacuum cleaner's filtration efficiency.

16. The vacuum cleaning system of claim 15, wherein the pad is circular.

17. The vacuum cleaning system of claim 15, wherein the filter is a foam filter or a HEPA filter.

18. The vacuum cleaning system of claim 15, wherein the pad has a thickness of 2 mm.

19. The vacuum cleaning system of claim 15, wherein the semisynthetic breathable felt material comprises a blend of natural and synthetic fibers.

20. The vacuum cleaning system of claim 19, wherein the natural fibers comprise wool and the synthetic fibers comprise polyester.
